(19) United States
(12) Patent Application Publication
(10) Pub. No.: US 2006/0182798 A1
(43) Pub. Date: Aug. 17, 2006

(54) Composition for gelatin coating, gelatin coating, and preparation using the same

(75) Inventors: Yoshiyuki Shimokawa, Akitakata-shi (JP); Sumihiro Shiraishi, Akitakata-shi (JP); Mika Mihara, Akitakata-shi (JP)

Correspondence Address: FISH & RICHARDSON PC, P.O. BOX 1022, MINNEAPOLIS, MN, 55440-1022, US

(73) Assignee: WAKUNAGA PHARMACEUTICAL CO., LTD.

(21) Appl. No.: 11/342,324

(22) Filed: Jan. 26, 2006

Related U.S. Application Data
(63) Continuation-in-part of application No. PCT/JP05/13916, filed on Jul. 29, 2005.

(30) Foreign Application Priority Data
Jul. 30, 2004 (JP) P2004-223745
Apr. 27, 2005 (JP) P2005-129968

(51) Int. Cl. A61K 9/64

(52) U.S. Cl. 424/456

(57) ABSTRACT

The present invention relates to a composition for gelatin coating and a gelatin coating characterized by containing a gelatin (A) and an inositol phosphate (B) represented by a general formula: C₆H₁₂₋ₙ.(H₂PO₄)ₙ (in the formula, n represents an integer from 1 to 6), and a preparation using the same.

The present invention claims priority on Japanese Patent Application No. 2004-223745 filed on Jul. 30, 2004, and Japanese Patent Application No. 2005-129968 filed on Apr. 27, 2005, the content of which is incorporated herein by reference. This application is a continuation in-part application of PCT/JP2005/013916 filed on Jul. 29, 2005.

### BACKGROUND OF THE INVENTION

1. Field of the Invention

The present invention relates to a composition for gelatin coating containing gelatin, a gelatin coating, and a preparation using the same.

2. Description of the Related Art

In the pharmaceutical or food field, preparations using gelatin for the coating thereof such as sugar-coated tablets, glue-coated tablets, and capsules are usually used in terms of safety, rapid solubility inside of the body, or the like. However, such preparations tend to decrease coating solubility over time, which relates to a bioavailability of an active ingredient, by, for example, cross-linking caused by interaction between a gelatin molecule and filled contents or decomposed matter thereof. This problem significantly occurs when the filled contents include galenicals, higher unsaturated fatty acids such as DHA, EPA, or the like, unsaturated fatty acid residue-containing oils and fats, minerals, mineral-containing yeasts, vitamin C, and the like, in particular.

In order to solve the above-mentioned problem, (1) addition of amino acids, citric acids, tartaric acids, or fumaric acids to gelatin coating (Japanese Examined Patent Application, Second Publication No. Sho 57-30088 and Japanese Unexamined Patent Application, First Publication No. Sho 59-39834), (2) addition of pullulan and polypeptide to gelatin coating (Japanese Unexamined Patent Application, First Publication No. Hei 05-65222), and the like have been proposed.

Moreover, it is known as a general problem of a soft capsule that the adhesion thickness of a coating jointed portion cannot be sufficiently obtained when the capsule is formed, and so the strength of the capsule becomes insufficient, and the leakage of the content of the capsule, the rupture of the capsule, or the like is cuased.

However, the above-mentioned prior art documents (1) and (2) have not yet resulted in sufficient prevention of a decrease of the solubility of the coating.

Also, effects of improvement in the strength of the soft capsule are not necessarily sufficient.

### SUMMARY OF THE INVENTION

The composition for gelatin coating of the present invention is characterized by containing a gelatin (A) and an inositol phosphate (B) represented by the general formula: C₆H₁₂₋ₙ.(H₂PO₄)ₙ (in the formula, n represents an integer from 1 to 6).

The gelatin coating of the present invention is characterized by containing a gelatin (A) and an inositol phosphate (B) represented by the general formula: C₆H₁₂₋ₙ.(H₂PO₄)ₙ (in the formula, n represents an integer from 1 to 6). In the gelatin coating of the present invention, it is preferable that the component (B) contains an inositol hexaphosphate.

The preparation of the present invention is characterized by including the gelatin coating of the present invention. The preparation of the present invention is particularly effective when a filled content thereof contains at least one selected from the group consisting of galenicals, unsaturated fatty acids, unsaturated fatty acid residue-containing oils and fats, minerals, mineral-containing yeasts, and vitamin C. Also, it is preferable that the preparation of the present invention has a filled content containing at least one selected from the group consisting of phospholipids, amino sugars, and organic acids. Moreover, it is preferable that the preparation of the present invention be any one selected from a capsule, a sugar-coated tablet, and a glue-coated tablet, and particularly preferable that the preparation be a soft capsule.

A production method of the soft capsule is characterized by including a production step of the composition for the gelatin coating containing at least the gelatin (A) and the inositol phosphate (B) represented by the general formula: C₆H₁₂₋ₙ.(H₂PO₄)ₙ (in the formula, n represents an integer from 1 to 6), and an encapsulation step in which the gelatin coating is produced from the composition for the gelatin coating and a content to be filled in the capsule is interposed between the gelatin coating followed by a pressure bonding.

Also, the above-mentioned production method may further include a step of preparing the content to be filled containing at least one selected from the group consisting of phospholipids, amino sugars, and organic acids.

A method for preventing insolubilization of the gelatin coating of the present invention is characterized by including formulating the inositol phosphate (B) represented by the general formula: C₆H₁₂₋ₙ.(H₂PO₄)ₙ (in the formula, n represents an integer from 1 to 6) into the gelatin.

### DETAILED DESCRIPTION OF THE INVENTION

The present invention has as an object thereof to provide a composition for a gelatin coating which can highly suppress a decrease of the solubility of the gelatin coating over time, particularly a composition for a gelatin coating for producing a soft capsule having a sufficient strength, and to provide the gelatin coating and the preparation using the same.

The inventors of the present invention have found that the above-mentioned problem can be solved by formulating an inositol phosphate into a gelatin coating, and has completed the present invention.

In the following, the present invention will be explained in detail.

“Composition for Gelatin Coating, and Gelatin Coating”

The composition for the gelatin coating of the present invention is a composition used for producing a coating of a preparation, particularly a capsule, a sugar-coated tablet, or a glue-coated tablet, and is characterized by containing at least a gelatin (A) and an inositol phosphate (B) represented by the general formula: C₆H₁₂₋ₙ.(H₂PO₄)ₙ (in the formula, n represents an integer from 1 to 6).

The gelatin coating of the present invention is characterized by containing the gelatin (A) and the inositol phosphate (B) represented by the general formula: C₆H₁₂₋ₙ⁻ (H₂PO₄)ₙ (in the formula, n represents an integer from 1 to 6).

The inventors of the present invention have found that formulation of an inositol phosphate into the gelatin coating significantly prevents a decrease of the solubility over time of a coating, the decrease being caused by, for example, cross-linking generated by interaction between a gelatin molecule and a filled content or decomposed matter thereof, and, in the case of a soft capsule, the formulation makes jointed portions of the coating to be bonded with a sufficient thickness, and so can give a sufficient strength to the capsule and prevent the leakage of the content from the jointed portions of the coating, the rupture of the capsule, or the like.

The gelatin (A), which is a main component, is not particularly limited, and conventional gelatins used for general capsules such as acid-treated gelatins, alkali-treated gelatins, amphoterically-treated gelatins, chemically-modified gelatins, or the like may be used. These may be used alone or in combination with two of more kinds thereof.

The gelatin is extracted after hydrolyzing a collagen, and, as a hydrolytic agent, the “acid-treated gelatin” uses an acid such as hydrochloric acid, sulfuric acid, or the like, the “alkali-treated gelatin” uses alkalis such as lime or the like, and the “amphoterically-treated gelatin” uses both the acid and the alkali. Moreover, the “chemically-modified gelatin” is one obtained by reacting an amino group of the gelatin with an organic acid such as succinic acid, phthalic acid, or the like. Among them, the acid-treated gelatin and the alkali-treated gelatin are preferably used.

The inositol phosphate (B) is one represented by the above-formula, and examples thereof include inositol monophosphate, inositol diphosphate, inositol triphosphate, inositol tetraphosphate, inositol pentaphosphate, and inositol hexaphosphate (phytic acid), which correspond to the number of phosphate group, n=1 to 6, respectively. These may be used alone or in combination with two or more kinds thereof.

Among them, it is preferable to use as the component (B) ones in which the number of phosphate groups is 3 to 6 (n=3 to 6), and particularly one (phytic acid) in which the number of phosphate groups is 6 (n=6), because effects of preventing a decrease of the solubility and increasing strength are excellently exhibited.

The amount of the inositol phosphate (B) added is not particularly limited. However, when the amount of the component (B) is extremely low, effects obtained by adding the component (effects of preventing a decrease of the solubility and increasing strength) may not be sufficiently exhibited, while when the amount is extremely high, particularly in the case of the soft capsule, a decrease of the strength of the capsule or adhesion between the capsules may be caused by a decrease of pH of the coating or a relative decrease of the amount of the gelatin. Accordingly, it is preferable that the amount of the inositol phosphate (B) in the coating of a capsule or a glue-coated tablet be 0.05 to 15% by mass, more preferably 1 to 10% by mass, and particularly preferably 2 to 8% by mass, with respect to the amount of the gelatin (A).

Also, it is preferable that the amount of the inositol phosphate (B) in the coating of a sugar-coated tablet be 0.1 to 150% by mass, more preferably 1 to 120% by mass, and particularly preferably 5 to 100% by mass, with respect to the amount of the gelatin (A).

The composition for the gelatin coating and the gelatin coating of the present invention may contain, in addition to the component (A) and the component (B), various additives generally used for coatings of capsules, sugar-coated tablets, or glue-coated tablets, such as, for example, plasticizers such as glycerin or sorbitol for components other than the component (B), such as amino acids, citric acids, or the like, antiseptics, coloring agents such as dyes or titanium oxide, organic acids, or the like, as needed.

The composition for the gelatin coating can be produced by mixing and dissolving at room temperature or while heating in water the component (A) and the component (B), and further various additives, as needed.

It is preferable that the gelatin coating be applied for coatings of capsules, sugar-coated tablets, or glue-coated tablets.

In brief explanation, the capsule coating can be produced by making the composition for the gelatin coating into a film, forming the film into a predetermined shape, and then drying. Although the content of water in the capsule coating after drying is not particularly limited, 5 to 20% by mass, particularly 7 to 15% by mass is preferable.

The timing of forming the capsule coating and filling the content thereof is different depending on types of the capsule. Although the present invention is applicable for both soft capsules and hard capsules, the soft capsules are particularly preferable.

The “soft capsule” is produced by formulating a plasticizer such as glycerin, sorbitol, or the like, and encapsulating a content to be filled by a film-like capsule coating which is relatively soft with increased plasticity, while or followed by forming it into a predetermined shape (encapsulation forming). The “hard capsule” is produced by directly filling a content to be filled into a capsule coating which is manufactured into a predetermined shape with relative hardness in advance or by slightly forming after filling.

The present invention is particularly effective in the case of the soft capsule which relatively tends to generate cross-linking caused by interaction between a gelatin molecule and the filled content or decomposed matter thereof.

In the case of the soft capsule, the capsule can be produced by, for example, manufacturing the composition for the gelatin coating into a film using a rotatory type soft capsule filler, supplying this film into a roll-metal mold from both left and right sides thereof, inserting while pressing a content to be filled into it just before punching it out into a predetermined shape, forming and drying.

In the case of the hard capsule, an immersion method can be used in which a stainless-steel mold-pin is immersed into an immersion fluid prepared by dissolving a gelatin in purified water while stirring and further adding inositol phophate to the solution, and the mold-pin is rotated to dry it.

The coating of a sugar-coated tablet can be formed in accordance with a generally known method, for example, one in which a tablet is placed in a coating pan, followed by protective-film coating (protective coating), under-coating (subcoating), middle-coating (smoothing), glazing (coloring), and drying. The composition for the gelatin coating is used for subcoating, and can be produced by dissolving while stirring in purified water the gelatin, the inositol phosphate, sugars (maltitol, erythritol, glucose, saccharose), and generally used additives such as plasticizers (such as glycerin).

The coating for a glue-coated tablet can be produced in accordance with a generally known method, for example, one in which the gelatin, the inositol phosphate (B), and a generally used plasticizer (such as glycerin) are mixed while stirring in purified water, and then dried. Between two sheets of the coating for the glue-coated tablet produced as described above, a tablet is placed and then compacted to produce a glue-coated tablet.

The composition for the gelatin coating and the gelatin coating of the present invention can significantly prevent a decrease of the solubility of the gelatin coating over time by formulating an inositol phosphate (B). Particularly, when the soft capsule is produced, the capsule can also increase the thickness of a jointed portion of the capsule coating, and so can exhibit a sufficient strength.

According to the technology of the present invention, since formulation of an inositol phosphate (B) into the composition for gelatin coating is simply required, the preparations such as capsules, sugar-coated tablets, glue-coated tablets, or the like, can be produced without changing conventional methods, which is preferable.

“Preparation”

A preparation of the present invention is characterized by including the above-mentioned gelatin coating of the present invention. It is preferable that the preparation of the present invention be a capsule, a sugar-coated tablet, or a glue-coated tablet. Although the capsule may be either a soft capsule or a hard capsule, a soft capsule is particularly preferable.

The form of the filled content is not particularly limited, it may be a liquid, suspension, paste, powder, granular, or the like. In the case of the granular, one having a coating formed by a coating agent may be used.

The preparation of the present invention can be used for various applications such as pharmaceuticals, quasi-drugs, health foods, general foods, cosmetics, or the like, and the constitution of the filled content is arbitrarily determined depending on the application of the preparation.

In the present invention, formulation of the inositol phosphate (B) into the gelatin coating can significantly inhibit a decrease of the solubility of the coating over time, and, particularly in the case of a soft capsule, can also give a sufficient strength to the capsule by increasing the thickness of a jointed portion of the capsule coating, and so effects can be demonstrated in spite of the constituent of the filled content.

Moreover, the inventors of the present invention have found that formulation of at least one selected from the group consisting of phospholipids, amino sugars, and organic acids in the filled content can decrease effects of the filled content on the gelatin coating and further prevent a decrease of the solubility of the coating over time.

The “phospholipid” is not particularly limited, and examples thereof include phosphatidyl serine, phosphatidylcholine (lecithin), phosphatidylinositol, phosphatidylethanolamine (cephalin), phosphatidylcardiolipin, phosphatidic acid, sphingomyelin, derivatives thereof, and the like. These may be used alone or in combination with two or more kinds thereof.

Among them, because effects of preventing a decrease of the solubility are excellently exhibited, phosphatidylethanolamine and derivatives thereof are preferable. Examples of the derivatives of phosphatidylethanolamine include a monoacyl type (lyzo type), alkenyl type, N-methyl type and N,N-dimethyl type in which base portions are methylated, N-acyl type, and the like, of a phosphatidylethanolamine that is a diacyl type.

As products containing at least one of the above-mentioned phospholipids, soybean phospholipid (soybean lecithin), yolk phospholipid (yolk lecithin), and the like are commercially available, and these are easily obtained and exhibit excellent effects, and so are preferably used. These may be purified products or crudely purified products, and also may be hydrogenated products or powdered products.

The “amino sugar” is not particularly limited, saccharides containing an amino group such as glucosamine, N-acetyl glucosamine, galactosamine, N-acetyl galactosamine, N-acetyl neuraminic acid, and the like, or polymers of glucosamine can be used, for example. Specific examples thereof include compounds having a structure in which a hydroxyl group of the saccharide is replaced with an amino group, such as chitosan and the derivatives thereof, polygalactosamine and derivatives thereof. Examples of chitosan and the derivatives thereof include low-molecular-weight chitosan, high-molecular-weight chitosan, chitosan oligosaccharide, and the like, and all those obtained by deacetylating chitins are preferably used. The amino sugars may be used alone or in combination with two or more kinds thereof.

The “organic acid” is not particularly limited, and examples thereof include amino acids such as tryptophan, aspartic acid, glutamic acid, glycine, phenylalanine, arginine, lysine, and the like, citric acid, succinic acid, fumaric acid, tartaric acid, lactic acid, malic acid, inositol phosphate, and the like. These may be used alone or in combination with two or more kinds thereof.

Among them, inositol phosphate and citric acid are preferable because they exhibit excellent inhibitory effects with respect to a decrease of the solubility. As inositol phosphate, although ones described above may be used, phytic acid is preferably used, in particular.

As described above, the filled content may contain at least one selected from the group consisting of phospholipids, amino sugars, and organic acids, and particularly preferably contains phospholipids and organic acids. Although the amount of these added is not particularly limited, extremely small addition cannot demonstrate sufficient effects of addition, while extremely large addition relatively decreases the amount of an active ingredient in the preparation, and so it is preferable that the total amount of addition with respect to the total amount of the filled content be 0.05 to 20% by mass, more preferably 0.1 to 10% by mass, and particularly 0.5 to 10% by mass.

Although the present invention can exhibit effects in spite of the constituent of the filled content, it is particularly effective when the filled content contains at least one selected from the group consisting of galenicals, unsaturated fatty acids, unsaturated fatty acid residue-containing oils and fats, minerals, mineral-containing yeasts, and vitamin C, because a problem in which the solubility of a gelatin coating decreases is significantly caused in the prior art.

The term “galenicals” refers to ones used as raw materials for pharmaceuticals (including Chinese medicinal drugs and folk medicines), perfumery, or spices, without modification or after being simply manufactured or prepared by, for example, cutting, crushing, and drying a part or all of animals, plants, and minerals without changing the nature thereof. In the capsule, the galenicals may be used in a form of powder, extract, essence, tincture, or the like.

Specific examples of the galenicals include plants such as Mallotus bark, Gambir, Aloe, Epimedium Herb, Fennel, Mume Fruit, Lindera Root, Bearberry Leaf, Turmeric, Rose Fruit, Acanthopanacis Cortex, Corydails Tuber, Rabdosiae Herb, Milkvetch Root, Scutellaria Root, Solomonseal Rhizome, Phellodendron Bark, Japanese Cherry Bark, Coptis Rhizome, Polygala Root, Phocae Testis et Penis, Sea Horse, Polygonum Tuber, Zedoary, Puerariae Radix, Valerianae Radix, Guarana, Glycyrrhiza, Platycodon Root, Immature Orange, Apricot Kernel, Barbary Wolfberry Fruit, Schizonepeta Spike, Cinnamon Bark, Cassia Seed, Gentian, Geranium Herb, Red Ginseng, Magnolia Bark, Oriental Bezoar, Acanthopanacis Cortex, Achyranthes Root, Evodia Fruit, Schisandra Fruit, Bupleurum Root, Asiasari Root, thyme, sage, Smilax Rhizome, Hawthorn Fruit, Gardenia Fruit, Cornus Fruit, Zanthoxylum Fruit, Zizyphus Seed, Dioscorea Rhizome, Rehmannia Root, civet, Peony Root, Cnidium Fruit, Plantago Herb, Houttuynia Herb, Amomum Seed, Ginger, Cardamon, Glossy Privet Fruit, Earthworm, Magnoliae Flos, Senega, Cnidium Rhizome, Peucedani Radix, Swertia Herb, Atractylodis Lanceae Rhizoma, Mori Cortex, Perillae Herba, Rhubarb, Zizyphi Fructus, Clove, Gambir Plant, Citrus Unshiu Peel, Capsicum, Japanese Angelicae Root, Tangshen, Peach Kernel, Bitter Orange Peel, Ipecac, Dodder Seed, Eucommia Bark, Nandinae Fructus, Nanbange, Cistanchis Herb, garlic, Ophiopogonis Tuber, Glehnia Root, Pinellia Tuber, Agkistrodon Japonicae, Atractylodis Rhizoma, Poria Sclerotium, Sinomenium Stem, Malaytea Scurfpea Fruit, Moutan Cortex, hop, Ephedra Herb, Actinidiae Fructi Galla, Muira Puama, Saussureae Radix, Coicis Semen, Longan Arillus, Gentianae Scabrae Radix, Scopolia Rhizome, Cervi Parvum Cornu, Chrysanthemum Flower, oat leaflet, Safflower, Salacia, Honeysuckle Stem, Ginseng (such as Panaxginseng, Panaxnotoginseng, or the like), Artemisia, green tea, herbs (such as ginkgo biloba, St John's Wort, chamomile, Piper methysticum, blueberry, bilberry, Serenoa repens, Salacia Oblonga, garcinia cambogia, rosemary, citrus, Vinca minor, echinacea, and the like), and the like, extracts, essences, or tinctures thereof;

powders obtained by drying and pulverizing without modification fungus (fruit body) such as Agaricus, Phellinus linteus, Ganoderma lucidum, Flammulina veluptipes, Schizophyllum commune, shiitake mushroom, Grifola frondosa, Chaga (Fuscoporia obliqua), mushroom, Lyophyllum decastes, Coriolus versicolor, Crepidotus mollis, bracket fungus, Cordyceps Sinensis Saccardo, Ganoderma lucidum, or the like, essences extracted from the fungus using hot water (which may include ethanol), essence powders, and the like;

extracts of animals, hydrolysates obtained by treating the extracts using acids, bases, or enzymes, ones collected from nests of animals (such as, for example, ox bile, chondroitin, glucosamine, collagen, propolis, and the like);

essences of fermented substances obtained by fermenting cereals, plants, marine products, or the like, using koji molds, red koji molds, lactic acid bacterias, acetic acid bacterias, Bacillus natto, yeasts, or the like;

Chinese medicinal drugs composed of a combination of galenicals such as, for example, Kakkonto (Puerariae Radix, Zizyphi Fructus, Peony Root, Ginger, Ephedra Herb, Cinnamon Bark, Glycyrrhiza), Tokishokuyakusan (Japanese Angelicae Root, Notopterygii Rhizome, Peony Root, Poria Sclerotium, Atractylodis Lanceae Rhizoma, Alismatis Rhizoma), Hachimijio (Rehmannia Root, Dioscorea Rhizome, Poria Sclerotium, Cinnamon Bark, Cornus Fruit, Alismatis Rhizoma, Moutan Cortex, Aconiti Tuber), Shoseiryuto (Ephedra Herb, Ginger, Cinnamon Bark, Schisandra Fruit, Peony Root, Glycyrrhiza, Asiasari Root, Pinellia Tuber), Bakumondoutou (Ophiopogonis Tuber, Oryzae Fructus, Ginseng, Pinellia Tuber, Zizyphi Fructus, Glycyrrhiza), Kami-Shoyosan (Japanese Angelicae Root, Atractylodis Lanceac Rhizoma, Bupleurum Root, Gardenia Fruit, Ginger, Peony Root, Poria Sclerotium, Moutan Cortex, Glycyrrhiza, Mentha Herb), and the like.

The filled content may include at least one of these galenicals.

The present invention is particularly effective when the filled content includes rosemary, citrus, blueberry, bilberry, propolis, Panaxnotoginseng, Panaxginseng, Agaricus, Phellinus linteus, shiitake mushroom, Ganoderma lucidum, and essences thereof, because effects in which a decrease of the coating solubility is prevented are large.

Although the “unsaturated fatty acid” is not particularly limited, the present invention is particularly effective when the filled content contains a long-chain unsaturated fatty acid having at least 14 carbon atoms, and more preferably 14 to 22 carbon atoms, because effects in which a decrease of the coating solubility is prevented are large.

Examples of the long-chain unsaturated fatty acid having at least 14 carbon atoms include DHA (docosahexaenoic acid), EPA (eicosapentaenoic acid), linoleic acid, arachidonic acid, Oleic acid, pinolenic acid, sciadonic acid, Jinoiperon acid, columbinic acid, conjugated linoleic acid, eleostearic acid, octadecenoic acid, octadecadienoic acid, docosenoic acid, ricinoleic acid, α-linolenic acid, γ-linolenic acid, behenic acid, phosphatidylcholine containing docosahexaenoic acid as a constituent fatty acid thereof (PC-DHA), phosphatidyl serine containing docosahexaenoic acid as a constituent fatty acid thereof (PS-DHA), phosphatidylcholine containing eicosapentaenoic acid as a constituent fatty acid (PC-EPA), phosphatidyl serine containing eicosapentaenoic acid as a constituent fatty acid thereof (PS-EPA), and the like. The filled content may contain at least one of these unsaturated fatty acids. The present invention is particularly effective when the filled content contains DHA and/or EPA.

The unsaturated fatty acid may be formulated in a form of isolated product of the unsaturated fatty acid or oil containing the same.

The “unsaturated fatty acid residue-containing oils and fats” refer to oils and fats in which at least one of fatty acid residues composing the oils and fats is an unsaturated fatty acid residue.

Examples of the oils containing at least one of the unsaturated fatty acid residue-containing oils and fats include vegetable oils such as soybean oil, rape seed oil, rice bran oil, cotton seed oil, sesame oil, sunflower oil, mustard oil, safflower oil, corn oil, peanut oil, olive oil, palm oil, coconut oil, and the like, and animal oils such as fish oil, whale oil, beef tallow, lard, milk fat, and the like. The filled content may contain at least one of these oils.

The present invention is particularly effective when the filled content contains a fish oil, because many unsaturated fatty acids (DHA and EPA) described above are also contained in the unsaturated fatty acid residue-containing oils and fats, and exhibit large effects in which a decrease of the coating solubility is prevented.

The “mineral” is not particularly limited, it referring to an inorganic substance useful from a nutritional standpoint, and examples thereof include calcium, phosphorus, iron, sodium, potassium, magnesium, zinc, selenium, copper, and the like. Among them, the present invention is particularly effective when calcium, phosphorus, iron, magnesium, zinc, selenium, or copper is contained, because use of polyvalent elements as a content filled in a conventional capsule significantly causes the insolubilization of the coating thereof, but the insolubilization can be inhibited in the capsule of the present invention. These may be used as inorganic or organic salts used for general foods.

The “mineral-containing yeast” is not particularly limited, examples thereof include magnesium-containing yeasts, zinc-containing yeasts, selenium-containing yeasts, iron-containing yeasts, copper-containing yeasts, and the like. The mineral-containing yeast is one in which a mineral is incorporated in the fungus body of a yeast.

The filled content may arbitrarily contain, in addition to the above-mentioned components, additives generally used for pharmaceuticals, foods, or the like, such as excipients, bonding agents, disintegrators, stabilizers, dispersants, coloring agents, flavoring compounds, medium-chain fatty acid monoglycerides, medium-chain fatty acid triglycerides, polyethylene glycols, surfactants (glycerin fatty acid esters, or the like), antioxidants (vitamin E, astaxanthin, catechin, or the like), or the like, as needed.

Since the preparation of the present invention includes the gelatin coating of the present invention, a decrease of the solubility over time is significantly prevented, and particularly in the case of a soft capsule, a sufficient strength is given to the capsule.

Particularly, formulation of at least one selected from the group consisting of phospholipids, amino sugars, and organic acids in the filled content can further prevent the decrease of the solubility, and is preferable.

### EXAMPLES

In the following, although test examples with respect to the present invention will be mentioned, the present invention is not limited to these.

Effect of Inhibiting Decrease of Coating Solubility

### Test Example 1

### Examples 1-1,1-2 and Comparative Examples 1-1 to 1-3

### Production of Gelatin Coating

100 g of gelatin and 45 g of glycerin were added to 100 g of purified water to make them absorb water and swell, followed by dissolving them at approximately 80° C. to produce a gelatin solution. To this solution, each organic acid of the kind in an amount as indicated in Table 1 (the concentration in the table represents the concentration (% by mass) with respect to the total amount of respective composition) was added, mixed, and then deaerated under reduced pressure to produce the respective composition for gelatin coating.

Each obtained composition was poured into a TLC plate, uniformly spread to a thickness of 1 mm, and then dried at 30° C. for 24 hours to obtain a film-like gelatin coating having a moisture content of approximately 9%. The coating obtained in respective example was cut into a 1 cm×1 cm small piece, and used for the following evaluation.

(Evaluation)

15 ml of DHA (fish oil) was poured in a screw tube, into which two pieces of each gelatin coating piece produced in the respective example were immersed, and stored at 50° C. After one or two day(s), the gelatin coating pieces were picked up, and the content adhered to the pieces was removed, followed by putting the pieces in 200 ml of hot water at 60° C. and stirring them with a stirrer bar for 2 minutes. The pieces were visually observed in the state of repose, and evaluated in accordance with the following criteria.

Criteria for Evaluation

(−): The gelatin coating pieces were completely dissolved, and the residue of the dissolved pieces was not recognized.

(±): The residue of the dissolved pieces was slightly recognized.

(+): The residue of the dissolved pieces was recognized in small amounts.

(++): The residue of the dissolved pieces was recognized in medium amounts.

(+++): The residue of the dissolved pieces was recognized in large amounts.

(++++): The gelatin coating pieces were not dissolved at all, and completely insolubilized.

(Results)

Results are shown in Table 1.

In Comparative Examples 1-1 to 1-3, the gelatin coating was already insolubilized completely two days after, while in Examples 1-1 and 1-2 in which inositol hexaphosphate (phytic acid) was added, the gelatin coating was completely dissolved even after two days. Accordingly, it is apparent that addition of inositol hexaphosphate significantly prevents a decrease of the solubility of gelatin coating over time.

TABLE 1
Effect of Inhibiting Decrease of Solubility of Gelatin Coating in DHA

| | | | Comparative | Comparative | Comparative |
|---|---|---|---|---|---|
| | Example | Example | Example | Example | Example |
| | 1-1 | 1-2 | 1-1 | 1-2 | 1-3 |
| Organic Acid | Inositol | Inositol | Additive-Free | Citric Acid | Citric Acid |
| (% by mass) | Hexaphosphate | Hexaphosphate | | (2%) | (4%) |
| | (2%) | (4%) | | | |
| First Day | − | − | ++ | + | + |
| Second Day | − | − | ++++ | ++++ | ++++ |

### Test Example 2

### Examples 2-1 to 2-15

(Production of Soft Capsule)

Each composition for gelatin coating composed of 45% by mass of gelatin, 18% by mass of glycerin, inositol phosphate of the kind in an amount as indicated in Table 2 (the concentration in the table represents the concentration (% by mass) with respect to the total amount of respective composition), and water (the remnant) was produced in the same way as that of Test Example 1, followed by deaerating and being left still for 10 hours to use for producing capsules.

Each component of the kind in an amount as indicated in the same table was filled using a rotatory type soft capsule filler (Oval Type 5). After filling, the coating of each capsule was dried at 27° C. and 50% or lower humidity till the moisture content in the coating of the capsule became 8% and so a soft capsule was obtained.

(Production of Sugar-Coated Tablet)

<Production of Inner Core Tablet>

1000 g of bilberry extract dried powder (Bilberon 25), 1200 g of crystalline cellulose, 700 g of milk sugar for direct tableting, 40 g of saccharose ester, 30 g of carmellose calcium, and 30 g of silica dioxide fine particle were mixed and granulated, followed by compacting using a tableting machine to produce tablets each having a weight of 200 mg.

<Formation of Protective Coating Layer>

250 g of hydroxypropylmethyl cellulose (TC-5) and 25 g of polyethylene glycol were dissolved in 1700 g of ethanol and 1125 g of purified water while stirring to produce a protective coating solution. In a coating device (High Coater 48), 2.8 kg of the inner core tablet was put, and spray-coated with the protective coating solution to form a protective coating layer till the weight of each tablet became approximately 10 mg.

<Formation of Middle-Coating Layer>

1600 g of white sugar, 60 g of gum arabic, 60 g of gelatin, 30 g of inositol hexaphosphate (phytic acid), 600 g of talc, and 900 g of calcium carbonate were dissolved in 800 g of purified water while heating to produce a middle-coating solution. The middle-coating solution was coated onto the protective coating layer of each tablet till the weight of the tablet became approximately 120 mg to form a middle-coating layer.

<Formation of Over-Coating Layer>

1750 g of white sugar was dissolved in 750 g of purified water while heating to produce an over-coating solution. The over-coating solution was coated onto the middle-coating layer of each tablet till the weight of the tablet became approximately 60 mg to form an over-coating layer. Moreover, grazing was carried out by adding 1.6 g of carnauba wax to produce a sugar-coated tablet having an approximate weight of 390 mg.

(Production of Glue-Coated Tablet)

<Production of Inner Core Tablet>

1000 g of bilberry extract dried powder (Bilberon 25), 1200 g of crystalline cellulose, 700 g of milk sugar for direct tableting, 40 g of saccharose ester, 30 g of carmellose calcium, and 30 g of silica dioxide fine particle were mixed and granulated, followed by compacting using a tableting machine to produce tablets each having a weight of 150 mg.

<Production of Gelatin Base (Coating)>

120 g of gelatin, 20 g of glycerin, and 5 g of phytic acid were added to 100 g of purified water, and then dissolved while heating, followed by deaerating under a reduced pressure to produce a gelatin coating solution. The gelatin solution was used to produce a gelatin base with a length of 100 mm and a width of 100 mm (thickness of approximately 1 mm) using a TLC plate fabrication device. Between two sheets of the produced gelatin base, the inner coat tablet was placed, followed by punching using a plate metal mold, and drying to produce a glue-coated tablet.

TABLE 2

| Ex- | Inositol Phosphate Added | Filled Content |
|---|---|---|
| ample | to Coating (% by mass) | (Amount) |
| 2-1 | Inositol Hexaphosphate (1%) | DHA (Fish Oil) (300 mg) |
| 2-2 | Inositol Hexaphosphate (2%) | DHA (Fish Oil) (300 mg) |
| 2-3 | Inositol Hexaphosphate (5%) | DHA (Fish Oil) (300 mg) |
| 2-4 | Inositol Hexaphosphate (8%) | DHA (Fish Oil) (300 mg) |
| 2-5 | Inositol Hexaphosphate (10%) | DHA (Fish Oil) (300 mg) |
| 2-6 | Inositol Hexaphosphate (4%) | Ganoderma Lucidum (130 mg) |
| | | Wheat Germ Oil (150 mg) |
| 2-7 | Inositol Hexaphosphate (4%) | Panaxnotoginseng (100 mg) |
| | | Propolis (150 mg) |
| 2-8 | Inositol Hexaphosphate (2%) | DHA (Fish Oil 1) (150 mg) |
| | | EPA (Fish Oil) (80 mg) |
| 2-9 | Inositol Hexaphosphate (2%) | Blueberry (120 mg) |
| | | DHA (Fish Oil) (150 mg) |
| 2-10 | Inositol Pentaphosphate (2%) | DHA (Fish Oil) (300 mg) |
| 2-11 | Inositol Triphosphate (2%) | DHA (Fish Oil) (300 mg) |
| 2-12 | Inositol Hexaphosphate (4%) | Vitamin C (100 mg) |
| | | Wheat Germ Oil (150 mg) |
| 2-13 | Inositol Hexaphosphate (4%) | Hemoferrum (100 mg) |
| | | Zinc-containing Yeast (50 mg) |
| | | Wheat Germ Oil (150 mg) |
| 2-14 | Inositol Hexaphosphate (4%) | PS-DHA (80 mg) |
| | | α-Linolenic Acid (200 mg) |
| 2-15 | Inositol Hexaphosphate (4%) | DHA (Fish Oil) (74 mg) |
| | | α-Linolenic Acid (140 mg) |
| | | Phosphatidyl Serine (70 mg) |

Effect of Giving Strength to Capsule

### Test Example 3

### Example 3 and Comparative Example 3

(Production of Gelatin Coating and Soft Capsule)

A gelatin solution (A) composed of 45% by mass of gelatin, 18% by mass of glycerin, 1% by mass of inositol hexaphosphate (phytic acid), and 36% by mass of water, and a gelatin solution (B) composed of 45% by mass of gelatin, 18% by mass of glycerin, and 37% by mass of water were respectively prepared, dissolved at 80° C., deaerated, and then left still for about 10 hours. Each gelatin coating with a thickness of 0.9. mm was formed from the gelatin solution (A) or (B) using a rotatory type soft capsule filler (Oval Type 5), 300 mg of DHA (fish oil) was filled inside of the respective coating to produce each soft capsule, and the capsule was then evaluated as described below.

(Evaluation)

Immediately after starting to fill each, filled capsules of No. 1, No. 4, and No. 7 (front, middle, back) were sampled from No. 1 to No. 7 capsule filler metal molds, and each content thereof was removed therefrom, followed by cutting in round slices to measure the thickness of the thinnest portion of the jointed portion thereof using a microscope with a scale for comparison. Moreover, after operating the filler for 1 hour using the respective gelatin solution, filled capsules were sampled in the same way as described above, and the thickness of the thinnest portion of the jointed portion thereof was measured for evaluation. Next, the adhesion ratio was calculated from the thickness of the jointed portion for evaluation.

(Results)

Results are shown in Table 3 and Table 4.

In comparison with Comparative Example 3 in which the capsules were produced using the gelatin solution (B), Example 3 in which the capsules were produced using the gelatin solution (A) containing an inositol hexaphosphate (phytic acid) demonstrated high adhesion ratio and it was confirmed that the strength of the capsules increased.

TABLE 3
Thickness of Jointed Portion

(Unit: mm)

| | | | Metal Mold | Metal Mold | Metal Mold | |
|---|---|---|---|---|---|---|
| | | | No. 1 | No. 4 | No. 7 | Average |
| | | | (Front Row) | (Middle Row) | (Back Row) | (Av) |
| Example 3 | Gelatin | Immediately | 0.45 | 0.46 | 0.43 | 0.45 |
| | Solution | After | | | | |
| | (A) | Filling | | | | |
| | | After 1 hour | 0.45 | 0.47 | 0.42 | 0.45 |
| Comparative | Gelatin | Immediately | 0.32 | 0.36 | 0.32 | 0.33 |
| Example 3 | Solution | After | | | | |
| | (B) | Filling | | | | |
| | | After 1 hour | 0.33 | 0.35 | 0.34 | 0.34 |

TABLE 4
Adhesion Ratio

(Unit: %)

| | | Immediately | | |
|---|---|---|---|---|
| | | After | After | Average |
| | | Filling | 1 hour | (Av) |
| Example 3 | Gelatin | 50.0 | 50.0 | 50 |
| | Solution | | | |
| | (A) | | | |
| Comparative | Gelatin | 36.7 | 37.8 | 37 |
| Example 3 | Solution | | | |
| | (B) | | | |

※ Adhesion Ratio (%) = (Average Adhesion Thickness (mm)/0.90 mm) × 100

As described above, the present invention can provide the composition for the gelatin coating of which the solubility over time is highly suppressed, and the gelatin coating and the preparation using the same. Particularly, when the preparation is a soft capsule, it is also possible to give a sufficient strength to the capsule.

In the above, although the preferable examples of the present invention are explained, the scope of the present invention is not limited to these examples. Addition, omission, or replacement of constituents or other modifications may be made without departing from the spirit of the present invention. The present invention is not limited to the above-mentioned explanation, but is limited to the scope of the appended claims.

What is claimed is:

1. A composition for a gelatin coating characterized by comprising a gelatin (A) and an inositol phosphate (B) represented by a general formula: C₆H₁₂₋ₙ.(H₂PO₄)ₙ (wherein n represents an integer from 1 to 6).

2. A gelatin coating characterized by comprising a gelatin (A) and an inositol phosphate (B) represented by a general formula: C₆H₁₂₋ₙ.(H₂PO₄)ₙ (wherein n represents an integer from 1 to 6).

3. A gelatin coating according to claim 2, characterized in that the component (B) comprises an inositol hexaphosphate.

4. A preparation characterized by comprising a gelatin coating according to claim 2.

5. A preparation according to claim 4, characterized in that a filled content thereof comprises at least one selected from the group consisting of galenicals, unsaturated fatty acids, unsaturated fatty acid residue-containing oils and fats, minerals, mineral-containing yeasts, and a vitamin C.

6. A preparation according to claim 4, characterized in that a filled content thereof comprises at least one selected from the group consisting of phospholipids, amino sugars, and organic acids.

7. A preparation according to claim 4, characterized by being any one selected from a capsule, a sugar-coated tablet, and a glue-coated tablet.

8. A preparation according to claim 4, characterized by being a soft capsule.

9. A production method of the preparation according to claim 8, characterized by comprising a production step of a composition for a gelatin coating containing at least a gelatin (A) and an inositol phosphate (B) represented by a general formula: C₆H₁₂₋ₙ.(H₂PO₄)ₙ (wherein n represents an integer from 1 to 6) and an encapsulation step in which the gelatin coating is produced from the composition for the gelatin coating and a content to be filled in the preparation is interposed between the gelatin coating followed by a pressure bonding.

10. A production method according to claim 9, characterized by further comprising a step of preparing the content to be filled containing at least one selected from the group consisting of phospholipids, amino sugars, and organic acids.

11. A method for preventing insolubilization of a gelating coating, characterized by comprising formulating an inositol phosphate (B) represented by a general formula: C₆H₁₂₋ₙ.(H₂PO₄)ₙ (wherein n represents an integer from 1 to 6) into a gelatin.
